(19) United States
(12) Patent Application Publication
(10) Pub. No.: US 2024/0026078 A1
(43) Pub. Date: Jan. 25, 2024

(54) Polymer taking the form of a pulverulent solid and having properties for thickening polar media

(71) Applicant: SOCIETE D'EXPLOITATION DE PRODUITS POUR LES INDUSTRIES CHIMIQUES SEPPIC, PARIS CEDEX 07 (FR)

(72) Inventors: Miruna BODOC, CASTRES (FR); Stéphane MONTEILLET, CASTRES (FR)

(21) Appl. No.: 18/256,553

(22) PCT Filed: Nov. 30, 2021
(86) PCT No.: PCT/EP2021/083620

§ 371 Date: Jun. 8, 2023

(30) Foreign Application Priority Data
Dec. 14, 2020 (FR) 2013174

(51) Int. Cl. C08G 69/10; C08J 3/12; C08J 3/24; A61Q 19/00; A61K 8/88

(52) U.S. Cl. CPC C08G 69/10; C08J 3/122; C08J 3/24; A61Q 19/00; A61K 8/88; C08J 2377/04

(57) ABSTRACT

Disclosed is a polymer (P) in the form of a pulverulent solid consisting of monomeric units derived from partially or totally solidified glutamic acid (GA), and monomer units of at least one crosslinking agent (AR) having at least two glycidyl functions.

The present invention relates to a polymer (P) in the form of a pulverulent solid, to the process for preparing said polymer and to a cosmetic composition comprising the polymer.

Polymers are widely used today in cosmetic formulations for topical use. They represent the second most widely used family of products in complex formulations of this type. Cosmetic compositions contain polar phases, for instance phases consisting of water, and in most cases require the use of rheology modifiers to increase the viscosity of these polar phases, and also to confer well-defined rheological behavior.

Among the polymers which modify the rheology of polar phases, mention may be made of natural polymers, for instance polysaccharides based on saccharides or saccharide derivatives or else synthetic polymers, of linear or branched, crosslinked or noncrosslinked, anionic, cationic or amphiphilic polyelectrolyte type. Predominantly present on the market of ingredients intended for cosmetic formulations, synthetic polymers have the property of being deployed, in the polar phase, under the effect of electrostatic repulsions due to the presence of negative and/or positive charges on the polymer backbone. These rheology-modifying agents provide both an increase in the viscosity of the polar phase, and also a degree of consistency and/or a stabilizing effect conferred on the cosmetic, dermocosmetic or demopharmaceutical formulation to be thickened.

In order to meet the needs of consumers and to improve the performance of cosmetic formulations, various recent scientific studies have reported the development of new, innovative and varied polymeric systems. The polymers used can then play different functional roles as:
film-forming agents,
rheology modifiers,
agents for stabilizing fatty phases in water-in-oil and oil-in-water emulsions,
agents for stabilizing solid particles (pigments or fillers),
agents conferring particular sensory properties (a soft feel, ease of uptake and application, freshness effect, etc.),
agents also having a direct impact on the appearance of the formula (transparent, translucent or opaque).

The polymers which modify the rheology of polar phases, and more particularly of aqueous phases, are mainly polyelectrolytes, resulting from the radical polymerization of (meth)acrylic type monomers, i.e. acrylic acid, methacrylic acid, esters derived from acrylic acid or methacrylic acid, or alternatively acrylamide or methacrylamide derivatives.

Developing new biobased and biodegradable rheology modifiers, that are as efficient as the synthetic polymers currently used, still constitutes a major challenge and a key issue for suppliers of cosmetic ingredients. Indeed, hitherto, the solutions mainly used for thickening aqueous phases involve ingredients originating from raw materials of petrochemical origin and notably acrylic acid or methacrylic acid and derivatives thereof.

Given the growing consumer concern for sustainable and responsible economy and development, replacing raw materials of petrochemical origin with raw materials of natural and renewable origin for preparing polymers is a priority research area.

To date, the literature describes the use of various natural polymers or polymers from renewable raw materials, the monomer units of which come from the family of sugars (glucose, arabinose, xylose, galactose, mannose, ribose, glucuronic acid, etc.) or from the family of amino acids (glutamic acid, aspartic acid, lysine, etc.). These polymers are mostly linear or branched depending on the plant from which they come or according to their manufacturing process.

As an example of a polymer of natural origin, mention may be made of polyglutamic acid (PGA), which is currently the subject of numerous research studies. It is a predominantly linear polymer and consists of glutamic acid (GA) monomer units. Glutamic acid is an amino acid characterized by an amine function in the α position and by two carboxylic acid functions (or carboxylates depending on the pH) in the α and γ positions (cf. chemical formula No. 1).

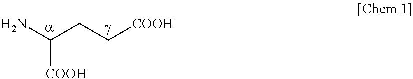

### Chemical Structure of Glutamic Acid (GA)

One of the ways to increase the branching of a synthetic or natural polymer or of a polymer of natural origin consists in performing crosslinking reactions. The purpose of crosslinking polymer chains is to connect together several polymer chains, which, when added to a polar phase, and more particularly to water, appear in the form of a three-dimensional network that is insoluble in water, but swellable with water, then leading to the production of an aqueous gel.

The preparation of crosslinked polymers may be performed in one or two steps according to the following two routes:
Route 1: In one step by reacting the monomers and the crosslinking agent during the polymerization reaction, or
Route 2: In at least two steps, the first of which consists in preparing the polymer, and the second consists in reacting the polymer with a crosslinking agent to obtain a crosslinked polymer.

Various reactions exist for the crosslinking of PGA, which make it possible to obtain polymers of natural origin with improved thickening properties in polar media, and notably in aqueous media.

Among the crosslinking agents known to be used in the PGA crosslinking reaction, polyepoxide derivatives are the most widely described since they make it possible to perform crosslinking processes under environmentally friendly conditions (moderate temperatures, reactions in aqueous media, and in the absence of harmful solvents).

However, the implementation of these processes requires diluting the PGA to high levels, which leads to the production of a composition in the form of an aqueous gel comprising, per 100% of its mass, a mass content less than or equal to 10% of a polymer, which is difficult for formulators to implement.

On this basis, a problem that arises is that of providing a composition comprising that is easy to formulate, comprising polymers of natural origin, the raw materials of which are renewable and which have thickening properties for polar media and more particularly for aqueous media.

One solution of the present invention is a polymer (P) in the form of a pulverulent solid comprising monomer units derived from partially or totally salified glutamic acid (GA), and monomer units of at least one crosslinking agent (XLA) bearing at least two glycidyl functions.

For the purposes of the invention, the term “pulverulent solid” means a solid consisting of fine particles that are sparingly bonded or not bonded together.

In the polymer (P) that is the subject of the present invention, the monomer units derived from partially or totally salified glutamic acid (GA) are linked together:
either via peptide or amide bonds involving the amine function of a glutamic acid (GA) monomer unit and the carboxylic acid (or carboxylate) function located in the alpha (a) position of a second glutamic acid (GA) monomer unit; the corresponding polymer is α-polyglutamic acid or PAGA (cf. chemical formula No. 2),

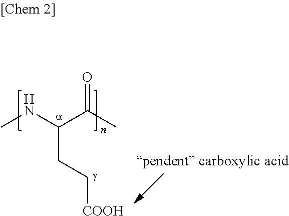

### Chemical Structure of α-Polyglutamic Acid or PAGA

or via amide bonds involving the amine function of a glutamic acid (GA) monomer unit and the carboxylic acid (or carboxylate) function located in the gamma (γ) position of
a second glutamic acid (GA) monomer unit; The corresponding polymer is γ-polyglutamic acid or PGGA (cf chemical formula No. 3),

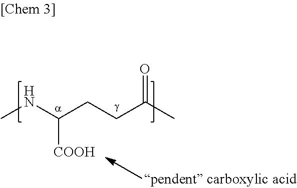

Chemical structure of γ-polyglutamic acid or PGGA.

In general, PGA may be prepared chemically according to peptide synthesis methods known to those skilled in the art, notably passing through selective protection, activation, coupling and deprotection steps. The coupling generally consists of a nucleophilic attack of the amine function of a glutamic acid monomer unit on an activated carboxylic acid function of another glutamic acid monomer unit.

PGGA can also be obtained via processes comprising at least one microbial fermentation step involving the use of at least one bacterial strain.

For the purposes of the present invention, in the polymer (P) as defined previously, the term “salified” indicates that the “pendent” carboxylic acid function present on each glutamic acid (GA) monomer unit of the polymer (in the γ position in the case of PAGA or in the a position in the case of PGGA) is in anionic or carboxylate form. The counterion of this carboxylate function is a cation derived, for example, from alkali metal salts such as sodium, potassium or salts of nitrogenous bases such as amines, lysine or monoethanolamine (HO—CH₂—CH₂—NH₂).

For the purposes of the present invention, the term “crosslinking agent (XLA)” denotes a chemical molecule whose structure makes it possible to bond covalently to at least two polymer chains.

For the purposes of the present invention, the term “crosslinking agent (XLA) bearing at least two glycidyl functions” denotes a crosslinking agent (XLA) as defined above, the molecular structure of which comprises at least two glycidyl units or functions of formula (I′):

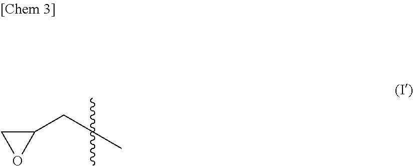 [SMILES: *CC1CO1]

The crosslinking of the polymer chains of the polymer (P) is performed according to a reaction between the terminal free amine function (—NH₂) and/or one or more “pendent” or terminal carboxylic (—COOH) or carboxylate (—COO⁻) functions present in the structure of said polymer (P), and at least one epoxide group present in the structure of the crosslinking agent (XLA) bearing at least two glycidyl functions.

The crosslinking agent (XLA) may be chosen from the members of the group consisting of:
monoethylene glycol diglycidyl ether of formula (I):

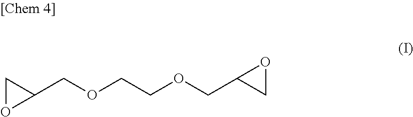 [SMILES: C(COCC1CO1)OCC1CO1]

the compound of formula (II):

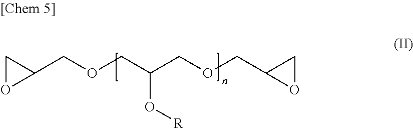

with R representing a hydrogen atom or the glycidyl radical

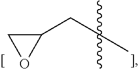 [SMILES: *CC1CO1]

and n representing an integer greater than or equal to 1 and less than or equal to 10;
when R represents a hydrogen atom and n is equal to 1, the compound is then glyceryl diglycidyl ether of formula (IIa):

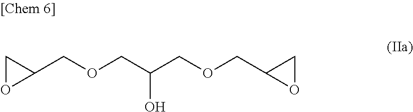 [SMILES: OC(COCC1CO1)COCC1CO1]

when R represents the glycidyl radical

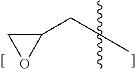 [SMILES: *CC1CO1]

and n is equal to 1, the compound is then glyceryl triglycidyl ether of formula (IIb):

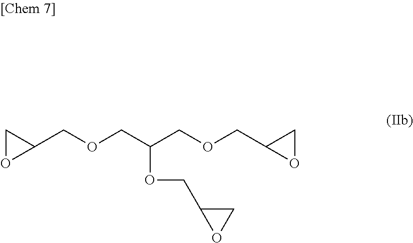 [SMILES: C(OCC1CO1)C(COCC1CO1)OCC1CO1]

when R represents a hydrogen atom and n is equal to 2, the compound is diglyceryl diglycidyl ether of formula (IIc):

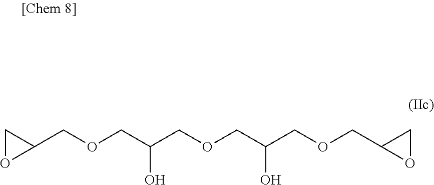 [SMILES: OC(COCC(O)COCC1CO1)COCC1CO1]

when R represents the glycidyl radical

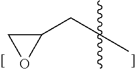 [SMILES: *CC1CO1]

and n is equal to 2, the compound is diglyceryl tetraglycidyl ether of formula (IId):

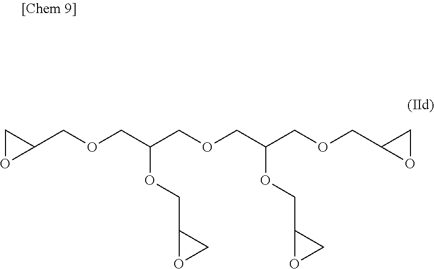 [SMILES: C(OCC(COCC1CO1)OCC1CO1)C(COCC1CO1)OCC1CO1]

1,3-propanediol diglycidyl ether of formula (III):

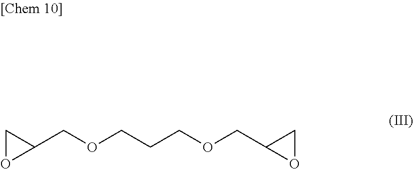 [SMILES: C(COCC1CO1)COCC1CO1]

1,2-propanediol diglycidyl ether of formula (IV):

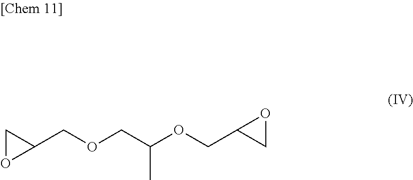

1,4-butanediol diglycidyl ether of formula (V):

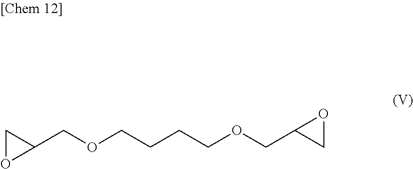 [SMILES: C(CCOCC1CO1)COCC1CO1]

1,2-butanediol diglycidyl ether of formula (VI):

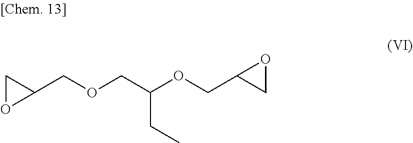

1,3-butanediol diglycidyl ether of formula (VII):

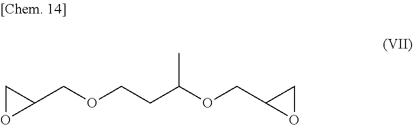

1,6-hexanediol diglycidyl ether of formula (VIII):

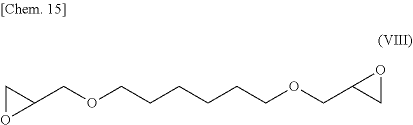 [SMILES: C(CCCOCC1CO1)CCOCC1CO1]

the compound of formula (IX):

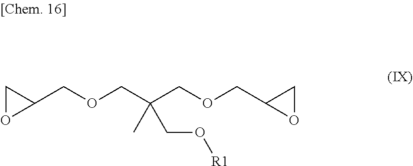

with R1 representing a hydrogen atom or the glycidyl radical

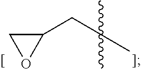 [SMILES: *CC1CO1]

when R1 represents a hydrogen atom, the compound is trimethylolethane diglycidyl ether of formula (IXa):

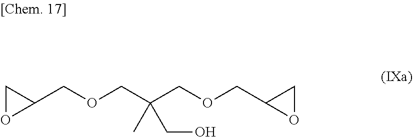 [SMILES: CC(CO)(COCC1CO1)COCC1CO1]

when R1 represents the glycidyl radical

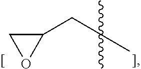 [SMILES: *CC1CO1]

the compound is trimethylolethane triglycidyl ether of formula (IXb):

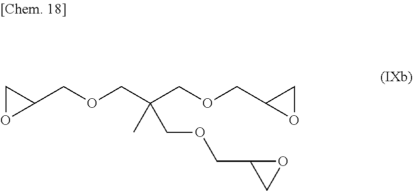 [SMILES: CC(COCC1CO1)(COCC1CO1)COCC1CO1]

the compound of formula (X):

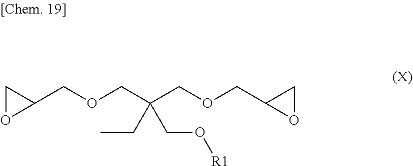

with R1 representing a hydrogen atom or the glycidyl radical

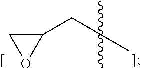 [SMILES: *CC1CO1]

when R1 represents a hydrogen atom, the compound is trimethylolpropane diglycidyl ether of formula (Xa):

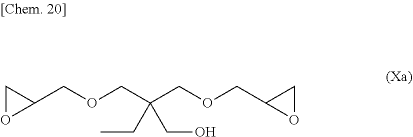 [SMILES: CCC(CO)(COCC1CO1)COCC1CO1]

when R1 represents the glycidyl radical

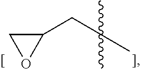 [SMILES: *CC1CO1]

the compound is trimethylolpropane triglycidyl ether of formula (Xb):

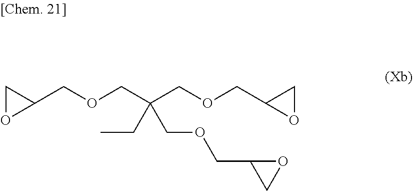 [SMILES: CCC(COCC1CO1)(COCC1CO1)COCC1CO1]

the compound of formula (XI):

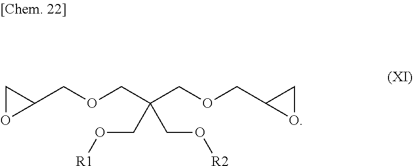

with R1 and R2 which are independent and represent a hydrogen atom or the glycidyl radical

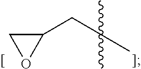 [SMILES: *CC1CO1]

when R1 and R2 each represent a hydrogen atom, the compound is pentaerythrityl diglycidyl ether of formula (XIa):

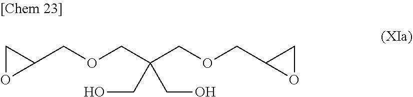 [SMILES: OCC(CO)(COCC1CO1)COCC1CO1]

when R1 represents a hydrogen atom and R2 represents the glycidyl radical

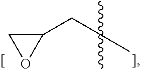 [SMILES: *CC1CO1]

the compound is pentaerythrityl triglycidyl ether of formula (XIb):

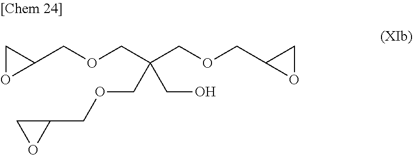 [SMILES: OCC(COCC1CO1)(COCC1CO1)COCC1CO1]

when R1 and R2 each represent the glycidyl radical

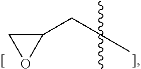 [SMILES: *CC1CO1]

the compound is pentaerythritol tetraglycidyl ether of formula (XIc):

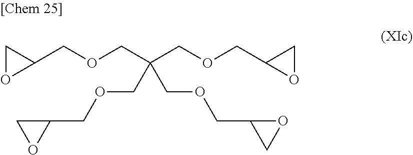 [SMILES: C(OCC(COCC1CO1)(COCC1CO1)COCC1CO1)C1CO1]

polyethylene glycol diglycidyl ether of formula (XII):

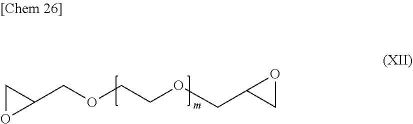

with m representing an integer greater than or equal to 2,
the compound of formula (XIII):

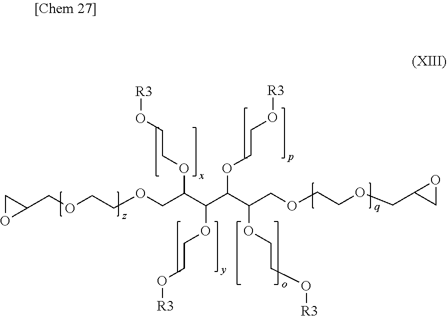

with R3 representing a hydrogen atom or the glycidyl radical

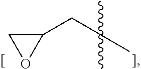 [SMILES: *CC1CO1]

and x, y, z, o, p and q, independently of each other, representing an integer greater than or equal to 2 and less than or equal to 10.

Moreover, the polymer (P) according to the invention may have one or more of the features below:
The polymer (P) is PGGA in acid form or partially or totally salified form,
per 100% of the mass of monomer units derived from partially or totally salified glutamic acid (GA), the crosslinking agent (XLA) represents from 0.5% to 10% by mass;
the polymer (P) also comprises a compound of formula (XIV):

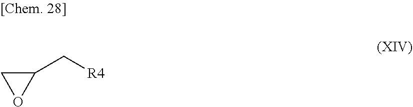

with R4 representing a linear or branched, saturated or unsaturated hydrocarbon-based radical optionally including at least one hydroxyl function and including from 6 to 22 carbon atoms,
R4 representing a hydrocarbon-based radical chosen from the elements of the group consisting of heptyl, octyl, nonyl, decyl, undecyl, undecenyl, dodecyl, tridecyl, tetradecyl, pentadecyl, hexadecyl, heptadecyl, octadecyl, hydroxyoctadecyl, oleyl, linoleyl, linolenyl, eicosyl and dodecosyl radicals,
In said polymer (P), per 100% of the mass of monomer units derived from partially or totally salified glutamic acid (GA), the compound of formula (XIV) represents from 1% to 50% by mass.

PGGA can exist in different conformational forms in solution in water. These forms depend on the inter- and intra-molecular hydrogen bonds and thus on the pH, the polymer concentration, the ionic strength of the solution, and also the temperature. The chains of the PGGA can thus adopt an a helical shape, a 13 sheet, aggregates or else be in a disordered and random state.

According to a particular aspect, the polymer (P) is in a helical conformation when it is present in a solution in a mass content of less than or equal to 0.1% and of which said solution has a pH value of less than or equal to 7.

According to a particular aspect, the polymer (P) is in sheet conformation when it is present in an aqueous solution in a mass content of less than or equal to 0.1% and of which said aqueous solution has a pH value above 7.

According to another particular aspect, per 100% of the mass of monomer units derived from partially or totally salified glutamic acid (GA), the crosslinking agent (XLA) represents from 0.5% to 8% by mass, and even more particularly from 0.5% to 5% by mass.

According to another particular aspect, R4 representing a hydrocarbon-based radical is chosen from the elements of the group consisting of n-octyl, n-nonyl, n-decyl, n-undecyl, n-undecenyl, n-dodecyl, n-tetradecyl, n-hexadecyl, n-octadecyl, 12-hydroxyoctadecyl, n-eicosyl and n-dodecosyl radicals.

A subject of the present invention is also a process for preparing a polymer (P) as defined previously, comprising the following three steps:
a) a step of preparing a polar phase (PP) comprising partially or totally salified PGA, at least one polar solvent (PS) and at least one crosslinking agent (XLA) comprising at least two glycidyl functions,
b) a step of adjusting the pH of the aqueous solution obtained in step a) to a pH of between 3 and 11, and
c) a step of drying by spraying the polar phase (PP) resulting from step b) so as to obtain the polymer (P).

As the case may be, the process according to the invention may have one or more of the following features:

In step a):
the polyglutamic acid used is PGGA,
all the monomer units constituting the PGA are derived from sodium glutamate, potassium glutamate, ammonium glutamate, calcium glutamate, magnesium glutamate or a mixture of these forms,
the polar phase (PP) comprise, per 100% of its mass: from 5% to 80% by mass of polyglutamic acid (PGA), from 0.025% to 8% by mass of the crosslinking agent (XLA), from 12% at 94.975% by mass of at least one polar solvent,
the polar solvent (PS) is chosen from the following list: water, methanol, ethanol, 1-propanol, 2-propanol, isobutanol, tert-butanol, 2-methyl-2-propanol, 1-butanol, 2-butanol, acetone, dimethyl ketone, diethyl ketone, tetrahydropyran, tetrahydrofuran, 2-methyltetrahydrofuran, 1,3-dioxane and 1,4-dioxane,
the polar phase (PP) also comprises at least one compound of formula (XIV):

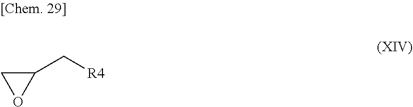

with R4 representing a linear or branched, saturated or unsaturated hydrocarbon-based radical optionally including at least one hydroxyl function and including from 6 to 22 carbon atoms,
R4 representing a hydrocarbon-based radical chosen from the elements of the group consisting of heptyl, octyl, nonyl, decyl, undecyl, undecenyl, dodecyl, tridecyl, tetradecyl, pentadecyl, hexadecyl, heptadecyl, octadecyl, hydroxyoctadecyl, oleyl, linoleyl, linolenyl, eicosyl and dodecosyl radicals,
the content of compound of formula (XIV) in the polar phase (PP) is, per 100% by mass of said polar phase (PP), between 0.05% and 35% by mass, it being understood that the sum of the mass proportions of the polyglutamic acid (PGA), of the crosslinking agent (XLA), of the polar solvent (PS) and of the compound of formula (XIV) is equal to 100%.
In step b):
the pH is between 4 and 10 and preferably between 5 and 7,
In step c):
the drying is performed with an atomizer using an air stream and a nozzle,
the inlet temperature of the air stream is between 80 and 180° C. and preferably between 100 and 170° C.

According to a particular aspect, the polar phase (PP) comprises, per 100% of its mass: from 5% to 70% by mass of the polyglutamic acid (PGA) of the polymer (P), from 0.025% to 7% by mass of the crosslinking agent (XLA), from 23% to 94.975% by mass of at least one polar solvent (PS).

According to an even more particular aspect, the polar phase (PP) comprises, per 100% of its mass: from 5% to 60% by mass of the polyglutamic acid (PGA) of the polymer (P), from to 6% by mass of the crosslinking agent (XLA), from 34% to 94.975% by weight of at least one polar solvent (PS).

According to another particular aspect, the polar solvent (PS) is chosen from the following list: water, ethanol, acetone, dimethyl ketone.

According to another even more particular aspect, the polar solvent (PS) is water. According to another particular aspect, R4 representing a hydrocarbon-based radical is chosen from the elements of the group consisting of n-octyl, n-nonyl, n-decyl, n-undecyl, n-undecenyl, n-dodecyl, n-tetradecyl, n-hexadecyl, n-octadecyl, 12-hydroxyoctadecyl, n-eicosyl and n-dodecosyl radicals.

According to another particular aspect, the content of compound of formula (XIV) in the polar phase (PP) is, per 100% by mass of said polar phase (PP), between 0.05% and 20% by mass, and even more particularly between 0.05% and 15% by mass, it being understood that the sum of the mass proportions of the polyglutamic acid (PGA), of the crosslinking agent (XLA), of the polar solvent (PS) and of the compound of formula (XIV) is equal to 100%.

Finally, the present invention also relates to a cosmetic composition for topical use (F) comprising, per 100% of its total mass, between 0.05% by mass and 10% by mass, more particularly between 0.1% by mass and 5% by mass, even more particularly between 0.1% by mass and 3% by mass of at least one polymer (P) as defined previously.

The term “topical” used in the definition of said composition (F) means that it is used by application to the skin, the hair, the scalp or the mucous membranes, whether this is a direct application, in the case of a cosmetic or dermocosmetic preparation, or an indirect application, for example in the case of a bodycare product in the form of a textile or paper wipe or of sanitary products intended to be in contact with the skin or the mucous membranes.

Said composition (F) is generally in the form of an aqueous or aqueous-alcoholic or water-glycol solution, in the form of a suspension, an emulsion, a microemulsion or a nanoemulsion, whether they are of water-in-oil, oil-in-water, water-in-oil-in-water or oil-in-water-in-oil type.

Said composition (F) as defined previously may be packaged in a bottle, in a device of pump-bottle type, in pressurized form in an aerosol device, in a device equipped with an openwork wall such as a grate or in a device equipped with a ball applicator (known as a roll-on).

In general, said composition (F) also includes excipients and/or active principles usually used in the field of formulations for topical use, in particular cosmetic or dermocosmetic formulations, such as thickening and/or gelling surfactants, stabilizers, film-forming compounds, hydrotropic agents, plasticizers, emulsifying and coemulsifying agents, opacifiers, nacreous agents, superfatting agents, sequestrants, chelating agents, antioxidants, fragrances, preserving agents, conditioning agents, bleaching agents intended for bleaching bodily hairs and the skin, active principles intended to provide a treating action with respect to the skin or the hair, sunscreens, pigments or mineral fillers, particles affording a visual effect or intended for the encapsulation of active principles, exfoliant particles or texturing agents.

As examples of foaming and/or detergent surfactants that may be combined with the polymer (P) in said composition (F) as defined previously, mention may be made of anionic, cationic, amphoteric or nonionic foaming and/or detergent surfactants.

Among the foaming and/or detergent anionic surfactants that may be combined with the polymer (P) in said composition (F) as defined previously, mention may be made of alkali metal salts, alkaline-earth metal salts, ammonium salts, amine salts or amino alcohol salts of alkyl ether sulfates, of alkyl sulfates, of alkylamido ether sulfates, of alkylaryl polyether sulfates, of monoglyceride sulfates, of α-olefin sulfonates, of paraffin sulfonates, of alkyl phosphates, of alkyl ether phosphates, of alkyl sulfonates, of alkylamide sulfonates, of alkylaryl sulfonates, of alkyl carboxylates, of alkylsulfosuccinates, of alkyl ether sulfosuccinates, of alkylamide sulfosuccinates, of alkyl sulfoacetates, of alkyl sarcosinates, of acyl isethionates, of N-acyl taurates, of acyl lactylates, of N-acylamino acid derivatives, of N-acyl peptide derivatives, of N-acyl protein derivatives, of N-acyl fatty acid derivatives.

Among the foaming and/or detergent amphoteric surfactants that may be combined with the polymer (P) in said composition (F) as defined previously, mention may be made of alkylbetaines, alkylamidobetaines, sultaines, alkylamidoalkylsulfobetaines, imidazoline derivatives, phosphobetaines, amphopolyacetates and amphopropionates.

Among the foaming and/or detergent cationic surfactants that may be combined with the polymer (P) in said composition (F) as defined previously, mention may be made in particular of quaternary ammonium derivatives.

Among the foaming and/or detergent nonionic surfactants that may be combined with the polymer (P) in said composition (F) as defined previously, mention may be made more particularly of alkylpolyglycosides including a linear or branched, saturated or unsaturated aliphatic radical, and including from 8 to 16 carbon atoms, for instance octyl polyglucoside, decyl polyglucoside, undecylenyl polyglucoside, dodecyl glucoside, tetradecyl polyglucoside, hexadecyl polyglucoside, 1,12-dodecanediyl polyglucoside; ethoxylated hydrogenated castor oil derivatives, for instance the product sold under the INCI name PEG-40 hydrogenated castor oil; polysorbates, for instance Polysorbate 20, Polysorbate 40, Polysorbate 60, Polysorbate 70, Polysorbate 80 and Polysorbate 85; coconut kernel amides; N-alkylamines.

As examples of thickening and/or gelling surfactants that may be combined with the polymer (P) in said composition (F) as defined previously, mention may be made of optionally alkoxylated alkylpolyglycoside fatty esters, for instance ethoxylated methylpolyglucoside esters, such as the PEG 120 methyl glucose trioleate and the PEG 120 methyl glucose dioleate sold, respectively, under the names Glutamate™ LT and Glutamate™ DOE120; alkoxylated fatty esters, such as the PEG 150 pentaerythrityl tetrastearate sold under the name Crothix™ DS53, the PEG 55 propylene glycol oleate sold under the name Antil™ 141; fatty-chain polyalkylene glycol carbamates, for instance the PPG-14 laureth isophoryl dicarbamate sold under the name Elfacos™ T211, the PPG-14 palmeth-60 hexyl dicarbamate sold under the name Elfacos™ GT2125.

Examples of thickening and/or gelling agents that may be combined with the polymer (P) in said composition (F) as defined previously, mention may be made of copolymers of AMPS and of alkyl acrylates, the carbon chain of which comprises between 4 and 30 carbon atoms and more particularly between 10 and 30 carbon atoms, linear, branched or crosslinked terpolymers of at least one monomer bearing a free, partially salified or totally salified strong acid function with at least one neutral monomer and at least one monomer of formula (XXX):

CH₂═C(R′₃)—C(═O)—[CH₂—CH₂—O]n′-R′₄ (XXX)
in which R′₃ represents a hydrogen atom or a methyl radical, R′₄ represents a linear or branched alkyl radical including from 8 to 30 carbon atoms and n′ represents a number greater than or equal to 1 and less than or equal to 50.

As examples of thickeners and/or gelling agents that may be combined with the polymer (P) in said composition (F) as defined previously, mention may be made of polysaccharides consisting only of monosaccharides, such as glucans or glucose homopolymers, glucomannoglucans, xyloglycans, galactomannans of which the degree of substitution (DS) of the D-galactose units on the main D-mannose chain is between 0 and 1, and more particularly between 1 and 0.25, such as galactomannans originating from cassia gum (DS=⅕), from locust bean gum (DS=¼), from tara gum (DS=⅓), from guar gum (DS=½) or from fenugreek gum (DS=1).

As examples of thickeners and/or gelling agents that may be combined with the polymer (P) in said composition (F) as defined previously, mention may be made of polysaccharides consisting of monosaccharide derivatives, such as sulfated galactans and more particularly carrageenans and agar, uronans and more particularly algins, alginates and pectins, heteropolymers of monosaccharides and uronic acids, and more particularly xanthan gum, gellan gum, gum arabic exudates and karaya gum exudates, or glucosaminoglycans.

As examples of thickeners and/or gelling agents that may be combined with the polymer (P) in said composition (F) as defined previously, mention may be made of cellulose, cellulose derivatives such as methyl cellulose, ethyl cellulose or hydroxypropyl cellulose, silicates, starch, hydrophilic starch derivatives, or polyurethanes.

As examples of stabilizers that may be combined with the polymer (P) in said composition (F) as defined previously, mention may be made of microcrystalline waxes, and more particularly ozokerite, mineral salts such as sodium chloride or magnesium chloride, silicone polymers such as polysiloxane polyalkyl polyether copolymers.

As examples of solvents that may be combined with the polymer (P) in said composition (F) as defined previously, mention may be made of water, organic solvents, such as glycerol, diglycerol, glycerol oligomers, ethylene glycol, propylene glycol, butylene glycol, 1,3-propanediol, 1,2-propanediol, hexylene glycol, diethylene glycol, xylitol, erythritol, sorbitol, water-soluble alcohols, such as ethanol, isopropanol or butanol, or mixtures of water and of said organic solvents.

As examples of thermal or mineral waters that may be combined with the polymer (P) in said composition (F) as defined previously, mention may be made of thermal or mineral waters having a mineralization of at least 300 mg/1, in particular Avene water, Vittel water, Vichy basin water, Uriage water, La Roche-Posay water, La Bourboule water, Enghien-les-Bains water, Saint-Gervais-les-Bains water, Neris-les-Bains water, Allevard-les-Bains water, Digne water, Maizieres water, Neyrac-les-Bains water, Lons-le-Saunier water, Rochefort water, Saint Christau water, Les Fumades water and Tercis-les-Bains water.

As examples of hydrotropic agents that may be combined with the polymer (P) in said composition (F) as defined previously, mention may be made of xylenesulfonates, cumenesulfonates, hexyl polyglucoside, 2-ethylhexyl polyglucoside or n-heptyl polyglucoside.

As examples of emulsifying surfactants that may be combined with the polymer (P) in said composition (F) as defined previously, mention may be made of nonionic surfactants, anionic surfactants or cationic surfactants.

As examples of emulsifying nonionic surfactants that may be combined with the polymer (P) in said composition (F) as defined previously, mention may be made of fatty acid esters of sorbitol, for instance the products sold under the names Montane™ 40, Montane™ 60, Montane™ 70, Montane™ 80 and Montane™ 85; compositions comprising glyceryl stearate and stearic acid ethoxylated with between 5 mol and 150 mol of ethylene oxide, for instance the composition comprising stearic acid ethoxylated with 135 mol of ethylene oxide and glyceryl stearate sold under the name Simulsol™ 165; mannitan esters, ethoxylated mannitan esters; sucrose esters; methyl glucoside esters; alkyl polyglycosides including a linear or branched, saturated or unsaturated aliphatic radical, and including from 14 to 36 carbon atoms, for instance tetradecyl polyglucoside, hexyldecyl polyglucoside, octadecyl polyglucoside, hexadecyl polyxyloside, octadecyl polyxyloside, eicosyl polyglucoside, dodecosyl polyglucoside, 2-octyldodecyl polyxyloside, 12-hydroxystearyl polyglucoside; compositions of linear or branched, saturated or unsaturated fatty alcohols including from 14 to 36 carbon atoms and of alkyl polyglycosides as described previously, for example the compositions sold under the names Montanov™ 68, Montanov™ 14, Montanov™ 82, Montanov™ 202, Montanov™ S, Montanov™ WO18, Montanov™ L, Fluidanov™ 20X and Easynov™.

As examples of anionic surfactants that may be combined with the polymer (P) in said composition (F) as defined previously, mention may be made of glyceryl stearate citrate, cetearyl sulfate, soaps such as sodium stearate or triethanolammonium stearate, or N-acyl derivatives of salified amino acids, for instance stearoyl glutamate.

As examples of emulsifying cationic surfactants that may be combined with the polymer (P) in said composition (F) as defined previously, mention may be made of amine oxides, quaternium-82 and the surfactants described in patent application WO 96/00719 and mainly those whose fatty chain comprises at least 16 carbon atoms.

As examples of opacifiers and/or nacreous agents that may be combined with the polymer (P) in said composition (F) as defined previously, mention may be made of sodium palmitate, sodium stearate, sodium hydroxystearate, magnesium palmitate, magnesium stearate, magnesium hydroxystearate, ethylene glycol monostearate, ethylene glycol distearate, polyethylene glycol monostearate, polyethylene glycol distearate and fatty alcohols including from 12 to 22 carbon atoms.

As examples of texturing agents that may be combined with the polymer (P) in said composition (F) as defined previously, mention may be made of N-acylamino acid derivatives, for instance lauroyl lysine sold under the name Aminohope™ LL, octenyl starch succinate sold under the name Dryflo™, myristyl polyglucoside sold under the name Montanov™ 14, cellulose fibers, cotton fibers, chitosan fibers, talc, sericite and mica.

As examples of deodorants that may be combined with the polymer (P) in said composition (F) as defined previously, mention may be made of alkali metal silicates; zinc salts, such as zinc sulfate, zinc gluconate, zinc chloride or zinc lactate; quaternary ammonium salts, such as cetyltrimethylammonium salts or cetylpyridinium salts; glycerol derivatives, such as glyceryl caprate, glyceryl caprylate or polyglyceryl caprate; 1,2-decanediol; 1,3-propanediol; salicylic acid; sodium bicarbonate; cyclodextrins; metal zeolites; Triclosan™; aluminum bromohydrate, aluminum chlorohydrates, aluminum chloride, aluminum sulfate, aluminum zirconium chlorohydrates, aluminum zirconium trichlorohydrate, aluminum zirconium tetrachlorohydrate, aluminum zirconium pentachlorohydrate, aluminum zirconium octachlorohydrate, aluminum sulfate, sodium aluminum lactate, or complexes of aluminum chlorohydrate and of glycol, such as the aluminum chlorohydrate and propylene glycol complex, the aluminum dichlorohydrate and propylene glycol complex, the aluminum sesquichlorohydrate and propylene glycol complex, the aluminum chlorohydrate and polyethylene glycol complex, the aluminum dichlorohydrate and polyethylene glycol complex or the aluminum sesquichlorohydrate and polyethylene glycol complex.

As examples of oils that may be combined with the polymer (P) in said composition (F) as defined previously, mention may be made of mineral oils such as liquid paraffin, liquid petroleum jelly, isoparaffins or white mineral oils; oils of animal origin such as squalene or squalane; plant oils, such as phytosqualane, sweet almond oil, coconut kernel oil, castor oil, jojoba oil, olive oil, rapeseed oil, groundnut oil, sunflower oil, wheat germ oil, corn germ oil, soybean oil, cotton oil, alfalfa oil, poppy oil, pumpkin oil, evening primrose oil, millet oil, barley oil, rye oil, safflower oil, candlenut oil, passionflower oil, hazelnut oil, palm oil, shea butter, apricot kernel oil, beauty-leaf oil, sisymbrium oil, avocado oil, calendula oil, oils derived from flowers or vegetables, ethoxylated plant oils; synthetic oils, for instance fatty acid esters such as butyl myristate, propyl myristate, isopropyl myristate, cetyl myristate, isopropyl palmitate, octyl palmitate, butyl stearate, hexadecyl stearate, isopropyl stearate, octyl stearate, isocetyl stearate, dodecyl oleate, hexyl laurate, propylene glycol dicaprylate, esters derived from lanolic acid, such as isopropyl lanolate, isocetyl lanolate, fatty acid monoglycerides, diglycerides and triglycerides, for instance glyceryl triheptanoate, alkylbenzoates, hydrogenated oils, poly(α-olefins), polyolefins such as poly(isobutane), synthetic isoalkanes, for instance isohexadecane, isododecane, perfluorinated oils; silicone oils, for instance dimethylpolysiloxanes, methylphenylpolysiloxanes, silicones modified with amines, silicones modified with fatty acids, silicones modified with alcohols, silicones modified with alcohols and fatty acids, silicones modified with polyether groups, epoxy-modified silicones, silicones modified with fluoro groups, cyclic silicones and silicones modified with alkyl groups. In the present patent application, the term “oils” refers to compounds and/or mixtures of compounds which are water-insoluble, and which have a liquid appearance at a temperature of 25° C.

As examples of waxes that may be combined with the polymer (P) in said composition (F) as defined previously, mention may be made of beeswax, carnauba wax, candelilla wax, ouricury wax, Japan wax, cork fiber wax, sugarcane wax, paraffin waxes, lignite waxes, microcrystalline waxes, lanolin wax; ozokerite; polyethylene wax; silicone waxes; plant waxes; fatty alcohols and fatty acids that are solid at room temperature; glycerides that are solid at room temperature. In the present patent application, the term “waxes” refers to compounds and/or mixtures of compounds which are water-insoluble, and which have a solid appearance at a temperature of greater than or equal to 45° C.

As examples of active principles that may be combined with the polymer (P) in said composition (F) as defined previously, mention may be made of vitamins and their derivatives, notably their esters, such as retinol (vitamin A) and its esters (for example retinyl palmitate), ascorbic acid (vitamin C) and its esters, sugar derivatives of ascorbic acid (such as ascorbyl glucoside), tocopherol (vitamin E) and its esters (such as tocopheryl acetate), vitamin B3 or B10 (niacinamide and its derivatives); compounds showing a lightening or depigmenting action on the skin, such as w-undecylenoyl phenylalanine sold under the name Sepiwhite™ MSH, Sepicalm™ VG, the glycerol monoester and/or the glycerol diester of ω-undecylenoyl phenylalanine, w-undecylenoyl dipeptides, arbutin, kojic acid, hydroquinone; compounds showing a calmative action, notably Sepicalm™ S, allantoin and bisabolol; anti-inflammatory agents; compounds showing a moisturizing action, such as urea, hydroxyureas, glycerol, polyglycerols, glycerol glucoside, diglycerol glucoside, polyglyceryl glucosides, xylityl glucoside; polyphenol-rich plant extracts, such as grape extracts, pine extracts, wine extracts or olive extracts; compounds showing a slimming or lipolytic action, such as caffeine or its derivatives, Adiposlim™, Adipoless™, fucoxanthin; N-acyl proteins; N-acyl peptides, such as Matrixyl™; N-acyl amino acids; partial hydrolyzates of N-acyl proteins; amino acids; peptides; total hydrolyzates of proteins; soybean extracts, for example Raffermine™; wheat extracts, for example Tensine™ or Gliadine™; plant extracts, such as tannin-rich plant extracts, isoflavone-rich plant extracts or terpene-rich plant extracts; extracts of freshwater or marine algae; marine plant extracts; marine extracts in general, such as corals; essential waxes; bacterial extracts; ceramides; phospholipids; compounds showing an antimicrobial action or a purifying action, such as Lipacide™ CBG, Lipacide™ UG, Sepicontrol™ A5, Octopirox™ or Sensiva™ SC50; compounds showing an energizing or stimulating property, such as Physiogenyl™, panthenol and its derivatives, such as Sepicap™ MP; antiaging active agents, such as Sepilift™ DPHP, Lipacide™ PVB, Sepivinol™, Sepivital™, Manoliva™, Phyto-Age™, Timecode™; Survicode™; antiphotoaging active agents; active agents which protect the integrity of the dermoepidermal junction; active agents which increase the synthesis of the components of the extracellular matrix, such as collagen, elastins or glycosaminoglycans; active agents which act favorably on chemical cell communication, such as cytokines, or physical cell communication, such as integrins; active agents which create a sensation of “heating” on the skin, such as activators of cutaneous microcirculation (such as nicotinic acid derivatives) or products which create a sensation of “coolness” on the skin (such as menthol and derivatives); active agents which improve cutaneous microcirculation, for example venotonics; draining active agents; active agents having a decongestant purpose, such as Ginkgo biloba, ivy, horse chestnut, bamboo, Ruscus, butcher's broom, Centella asiatica, fucus, rosemary or willow extracts; agents for tanning or browning the skin, for example dihydroxyacetone (DHA), erythrulose, mesotartaric aldehyde, glutaraldehyde, glyceraldehyde, alloxan or ninhydrin, plant extracts, for example extracts of red woods of the genus Pterocarpus and of the genus Baphia, such as Pteropcarpus santalinus, Pterocarpus osun, Pterocarpus soyauxii, Pterocarpus erinaceus, Pterocarpus indicus or Baphia nitida, such as those described in the European patent application EP 0 971 683; agents known for their action in facilitating and/or accelerating tanning and/or browning of human skin, and/or for their action in coloring human skin, for example carotenoids (and more particularly β-carotene and γ-carotene), the product sold under the brand name Carrot Oil (INCI name: Daucus carrota, Helianthus annuus sunflower oil) by the company Provital, which contain carotenoids, vitamin E and vitamin K; tyrosine and/or its derivatives, known for their effect on accelerating the tanning of human skin in combination with exposure to ultraviolet radiation, for example the product sold under the brand name SunTan Accelerator™ by the company Provital, which contains tyrosine and riboflavins (vitamin B), the complex of tyrosine and of tyrosinase sold under the brand name Zymo Tan Complex by the company Zymo Line, the product sold under the brand name MelanoBronze™ (INCI name: Acetyl Tyrosine, Monk's pepper extract (Vitex agnus-castus)) by the company Mibelle, which contains acetyl tyrosine, the product sold under the brand name Unipertan VEG-24/242/2002 (INCI name: Butylene Glycol and Acetyl Tyrosine and Hydrolyzed Vegetable Protein and Adenosine Triphosphate) by the company Unipex, the product sold under the brand name Try-Excell™ (INCI name Oleoyl tyrosine and Luffa cylindrica (seed) oil and oleic acid) by the company Sederma, which contains extracts of marrow seeds (or loofah oil), the product sold under the brand name Actibronze™ (INCI name: Hydrolyzed wheat protein and acetyl tyrosine and copper gluconate) by the company Alban Muller, the product sold under the brand name Tyrostan™ (INCI name: Potassium caproyl tyrosine) by the company Synerga, the product sold under the brand name Tyrosinol (INCI name: Sorbitan Isostearate, Glyceryl Oleate, Caproyl Tyrosine) by the company Synerga, the product sold under the brand name InstaBronze™ (INCI name: Dihydroxyacetone and Acetyl Tyrosine and Copper Gluconate) by the company Alban Muller, the product sold under the brand name Tyrosilane (INCI name Methylsilanol and Acetyl Tyrosine) by the company Exymol; peptides known for their effect in activating melanogenesis, for example the product sold under the brand name Bronzing SF Peptide Powder (INCI name: Dextran and Octapeptide-5) by the company Infinitec Activos, the product sold under the brand name Melitane (INCI name: Glycerin and Aqua and Dextran and Acetyl Hexapeptide-1) comprising acetyl hexapeptide-1 known for its α-MSH agonist action, the product sold under the brand name Melatimes Solutions™ (INCI name Butylene glycol, Palmitoyl Tripeptide-40) by the company Lipotec, sugars and sugar derivatives, for example the product sold under the brand name Tanositol™ (INCI name: inositol) by the company Provital, the product sold under the brand name Thalitan™ (or Phycosaccharide™ AG) by the company CODIF International (INCI name: Aqua and Hydrolyzed algin (Laminaria digitata) and magnesium sulfate and manganese sulfate) containing an oligosaccharide of marine origin (guluronic acid and mannuronic acid chelated with magnesium and manganese ions), the product sold under the brand name Melactiva™ (INCI name: Maltodextrin, Mucuna pruriens Seed Extract) by the company Alban Muller, compounds rich in flavonoids, for example the product sold under the brand name Biotanning (INCI name: Hydrolyzed citrus Aurantium dulcis fruit extract) by the company Silab and known to be rich in lemon flavonoids (of the hesperidin type); agents intended for treating head hair and/or bodily hair, for example agents for protecting the melanocytes of the hair follicle, intended to protect said melanocytes against cytotoxic agents responsible for the senescence and/or apoptosis of said melanocytes, such as mimetics of DOPAchrome tautomerase activity, chosen from those described in the European patent application published under the number EP 1 515 688 A2, synthetic SOD mimetic molecules, for example manganese complexes, antioxidant compounds, for example cyclodextrin derivatives, siliceous compounds derived from ascorbic acid, lysine or arginine pyrrolidone carboxylate, combinations of mono- and diesters of cinnamic acid and of vitamin C, and more generally those mentioned in the European patent application published under the number EP 1 515 688 A2.

As examples of antioxidants that may be combined with the polymer (P) in said composition (F) as defined previously, mention may be made of EDTA and salts thereof, citric acid, tartaric acid, oxalic acid, BHA (butylhydroxyanisole), BHT (butylhydroxytoluene), tocopherol derivatives such as tocopheryl acetate, mixtures of antioxidant compounds such as Dissolvine™ GL 47S sold by the company AkzoNobel under the INCI name Tetrasodium Glutamate Diacetate.

As examples of sunscreens that may be combined with the polymer (P) in said composition (F) as defined previously, mention may be made of all those appearing in the Cosmetic Directive 76/768/EEC, amended, Annex VII.

Among the organic sunscreens that may be combined with the polymer (P) in said composition (F) as defined previously, mention may be made of the family of benzoic acid derivatives, for instance para-aminobenzoic acids (PABA), notably monoglyceryl esters of PABA, ethyl esters of N,N25-propoxy PABA, ethyl esters of N,N-diethoxy PABA, ethyl esters of N,N-dimethyl PABA, methyl esters of N,N-dimethyl PABA and butyl esters of N,N-dimethyl PABA; the family of anthranilic acid derivatives, for instance homomenthyl-N-acetyl anthranilate; the family of salicylic acid derivatives, for instance amyl salicylate, homomenthyl salicylate, ethylhexyl salicylate, phenyl salicylate, benzyl salicylate and p-isopropanolphenyl salicylate; the family of cinnamic acid derivatives, for instance ethylhexyl cinnamate, ethyl 4-isopropylcinnamate, methyl 2,5-diisopropylcinnamate, p-methoxypropyl cinnamate, p-methoxyisopropyl cinnamate, p-methoxyisoamyl cinnamate, p-methoxyoctyl cinnamate (p-methoxy-2-ethylhexyl cinnamate), p-methoxy-2-ethoxyethyl cinnamate, p-methoxycyclohexyl cinnamate, ethyl-α-cyano-P-phenyl cinnamate, 2-ethylhexyl-α-cyano-β-phenyl cinnamate or glyceryl di-para-methoxymono-2-ethylhexanoyl cinnamate; the family of benzophenone derivatives, for instance 2,4-dihydroxybenzophenone, 2,2′-dihydroxy-4-methoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl 4′-phenylbenzophenone-2-5-carboxylate, 2-hydroxy-4-n-octyloxybenzophenone, 4-hydroxy-3-carboxybenzophenone; 3-(4′-methylbenzylidene)-d,l-camphor, 3-(benzylidene)-d,l-camphor, camphor benzalkonium methosulfate; urocanic acid, ethyl urocanate; the family of sulfonic acid derivatives, for instance 2-phenylbenzimidazole-5-sulfonic acid and salts thereof; the family of triazine derivatives, for instance hydroxyphenyltriazine, ethylhexyloxyhydroxyphenyl)-4-methoxyphenyltriazine, 2,4,6-trianillino(p-carbo-2′-ethylhexyl-1′-oxy)-1,3,5-triazine, 4,4-((6-(((1,1-dimethylethyl)amino)carbonyllphenyl)amino)-1,3,5-triazine-2,4-diyldiimino)bis (2-ethylhexyl) benzoate, 2-phenyl-5-methylbenzoxazole, 2,2′-hydroxy-5-methylphenylbenzotriazole, 2-(2′-hydroxy-5′-t-octylphenyl)benzotriazole, 2-(2′-hydroxy-5′-methyphenyl)benzotriazole; dibenzazine; dianisoylmethane, 4-methoxy-4″-t-butylbenzoylmethane; 5-(3,3-dimethyl-2-norbornylidene)-3-pentan-2-one; the family of diphenylacrylate derivatives, for instance 2-ethylhexyl 2-cyano-3,3-diphenyl-2-propenoate, ethyl 2-cyano-3,3-diphenyl-2-propenoate; the family of polysiloxanes, for instance benzylidene siloxane malonate.

Among the mineral sunscreens, also known as “mineral sunblocks”, that may be combined with the polymer (P) in said composition (F) as defined previously, mention may be made of titanium oxides, zinc oxides, cerium oxide, zirconium oxide, yellow, red or black iron oxides, and chromium oxides. These mineral sunblocks may or may not be micronized, may or may not have been subjected to surface treatments and may optionally be in the form of aqueous or oily predispersions.

### EXAMPLES

The examples that follow illustrate the invention without, however, limiting it.

### Example 1: Preparation of a Sodium PGGA According to the Invention Crosslinked with Polyethylene Glycol Diglycidyl Ether and Atomized

The synthetic process comprises the following four steps:

Step 1): Preparation of an Aqueous Sodium PGGA Gel:

200 grams of demineralized water are stirred with a Rayneri™ brand mechanical stirrer equipped with a deflocculating-type rotor, and 20 grams of PGGA, sold by the company Lubon under the name Cosmetic Grade PGGA, are then slowly added into the vortex.

Step 2): pH Adjustment:

The pH of the mixture from step 1) is adjusted to a value of between 5.5 and 6.0 using aqueous 5M HCl solution (temperature=20° C.).

Step 3): Addition of the Crosslinking Agent:

1.2 grams of polyethylene glycol diglycidyl ether (PEGDGE) with an average molecular weight of the order of 500 g/mol are added to the mixture of step 2).

Step 4): Atomization:

The aqueous solution obtained in step 3) is concentrated by passage through an atomizer whose operating parameters are as follows:
Solution introduction rate=4 ml/min,
Spraying nozzle pressure=1.5 bar,
Circulating air temperature=150° C.,

Composition (E1) is finally isolated in the form of a pulverulent powder.

### Example 2: Preparation of a Sodium PGGA According to the Invention Crosslinked with 1,4-Butanediol Diglycidyl Ether and Atomized

The synthetic process of Example 1 is reproduced, replacing the 1.2 grams of polyethylene glycol diglycidyl ether (PEGDGE) with 0.48 gram of 1,4-butanediol diglycidyl ether sold under the name Erisys™ GE 21 by the company Emerald.

The composition (E2) obtained is finally isolated in the form of a pulverulent powder.

### Evaluation of the Crosslinked and Atomized Sodium PGGAs Obtained in the Two Preceding Examples:

The evaluation of compositions (E1) and (E2) (crosslinked and atomized sodium PGGAs), obtained in the two preceding examples was performed as follows:
In a 400 ml high-sided beaker, dispersion of 4 grams of the compositions to be tested in 196 grams of water with stirring using a Rayneri™ brand mechanical stirrer equipped with a deflocculating-type rotor, until a homogeneous gel is obtained,
Measurement of the dynamic viscosity using a Brookfield RVT viscometer, speed 5, choosing the appropriate spindle,
Addition of 0.1% of NaCl to the gel obtained previously, stirring with a Rayneri™ brand mechanical stirrer equipped with a deflocculating-type rotor, until the mixture is homogeneous,
Measurement of the dynamic viscosity using the same viscometer as previously, again choosing the appropriate spindle.

A control was produced from non-crosslinked PGGA sold by the company Lubon under the name Cosmetic Grade PGGA.

The measured dynamic viscosity values are collated in the table below:

TABLE 1

| | | Viscosity in | Viscosity in |
|---|---|---|---|
| | | mPa · s of | mPa · s of the gel |
| | Nature of | the gel | containing |
| | crosslinking | containing 2% | 2% polymer + |
| Composition | agent | polymer | 0.1% NaCl |
| Control^{a)} | No | 176 (spindle 2) | 128 (spindle 2) |
| Composition | PEGDGE | 30 400 (spindle 5) | 9320 (spindle 3) |
| (E1) | | | |
| Composition | Erisys ™ GE 21 | 41 520 (spindle 5) | / |
| (E2) | | | |

^{a)}Non-crosslinked PGGA sold by the company Lubon under the name Cosmetic Grade PGGA

Dynamic viscosities of aqueous gels obtained with compositions (E1) and (E2).

The control sodium PGGA gels (non-crosslinked and non-atomized) have viscosities of between 100 and 200 mPa·s in the presence or absence of NaCl.

In the absence of NaCl, the crosslinked and atomized sodium PGGA gels have viscosities respectively equal to 30 400 mPa·s in the case where the crosslinking agent is PEGDGE and 41 520 mPa·s with Erisys™ GE 21 as crosslinking agent.

In the presence of NaCl, the viscosity of the sodium PGGA gel crosslinked with PEGDGE and atomized is equal to 9320 mPa·s.

Compositions (E1) and (E2) according to the invention thus make it possible to thicken aqueous phases.

1. A polymer (P) in the form of a pulverulent solid comprising monomer units derived from partially or totally salified glutamic acid (GA), and monomer units of at least one crosslinking agent (XLA) bearing at least two glycidyl functions.

2. The polymer (P) as claimed in claim 1, wherein the crosslinking agent (XLA) is chosen from the members of the group consisting of:
monoethylene glycol diglycidyl ether of formula (I): 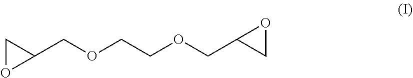 [SMILES: C(COCC1CO1)OCC1CO1]
the compound of formula (II): 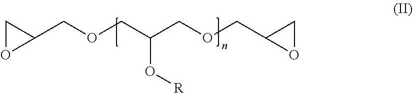
with R representing a hydrogen atom or the glycidyl radical 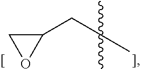 [SMILES: *CC1CO1]
and n representing an integer greater than or equal to one and less than or equal to 10;
1,3-propanediol diglycidyl ether of formula (III): 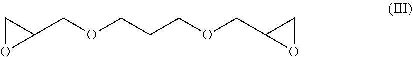 [SMILES: C(COCC1CO1)COCC1CO1]
1,2-propanediol diglycidyl ether of formula (IV): 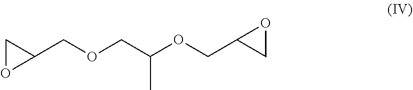
1,4-butanediol diglycidyl ether of formula (V): 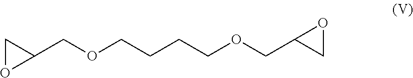 [SMILES: C(CCOCC1CO1)COCC1CO1]
1,2-butanediol diglycidyl ether of formula (VI): 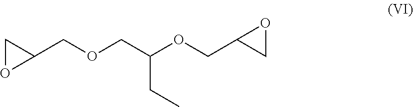
1,3-butanediol diglycidyl ether of formula (VII): 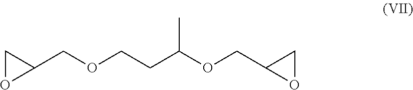
1,6-hexanediol diglycidyl ether of formula (VIII): 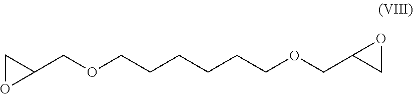 [SMILES: C(CCCOCC1CO1)CCOCC1CO1]
the compound of formula (IX): 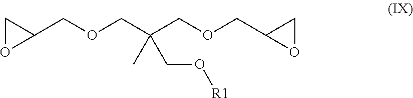
with R1 representing a hydrogen atom or the glycidyl radical 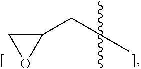 [SMILES: *CC1CO1]
the compound of formula (X): 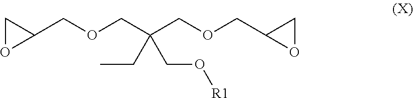
with R1 representing a hydrogen atom or the glycidyl radical 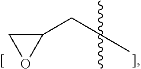 [SMILES: *CC1CO1]
the compound of formula (XI): 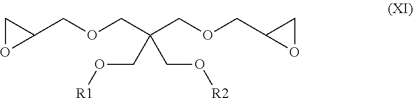
with R1 and R2 which are independent and represent a hydrogen atom or the glycidyl radical 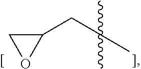 [SMILES: *CC1CO1]
the compound of formula (XII): 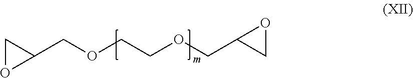
with m representing an integer greater than or equal to 2,
the compound of formula (XIII): 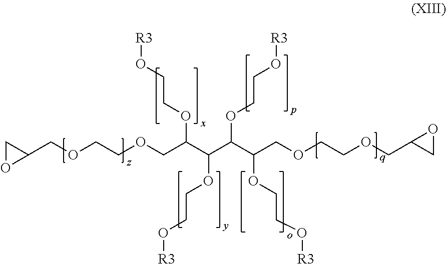
with R3 representing a hydrogen atom or the glycidyl radical 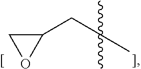 [SMILES: *CC1CO1]
and x, y, z, o, p and q, independently of each other, representing an integer greater than or equal to 2 and less than or equal to 10.

3. The polymer (P) as defined in either of claim 1, wherein the polyglutamic acid (PGA) is γ-polyglutamic acid or PGGA.

4. The polymer (P) as claimed in claim 1, wherein, per 100% of the mass of monomer units derived from partially or totally salified glutamic acid (GA), the crosslinking agent (XLA) represents from 0.5% to 10% by mass.

5. The polymer (P) as claimed in claim 1, wherein said polymer also comprises a compound of formula (XIV): 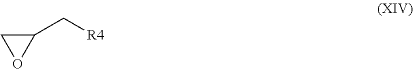
with R4 representing a linear or branched, saturated or unsaturated hydrocarbon-based radical optionally including at least one hydroxyl function and including from 6 to 22 carbon atoms.

6. The polymer (P) as claimed in claim 5, wherein R4 represents a hydrocarbon-based radical chosen from the elements of the group consisting of heptyl, octyl, nonyl, decyl, undecyl, undecenyl, dodecyl, tridecyl, tetradecyl, pentadecyl, hexadecyl, heptadecyl, octadecyl, hydroxyoctadecyl, oleyl, linoleyl, linolenyl, eicosyl and dodecosyl radicals.

7. The polymer (P) as claimed in claim 5, wherein per 100% of the mass of monomer units derived from partially or totally salified glutamic acid (GA), the compound of formula (XIV) represents from 1% to 50% by mass.

8. A process for preparing a polymer (P) as defined in claim 1, comprising:
a) a step of preparing a polar phase (PP) comprising partially or totally salified polyglutamic acid, at least one polar solvent (PS) and at least one crosslinking agent (XLA) comprising at least two glycidyl functions,
b) a step of adjusting the pH of the aqueous solution obtained in step a) to a pH of between 3 and 11,
c) a step of drying by spraying the polar phase (PP) resulting from step b) so as to obtain the polymer (P).

9. The process as claimed in claim 8, wherein in step a), the polyglutamic acid is PGGA.

10. The process as claimed in claim 9, wherein in step a), all of the monomer units constituting the polyglutamic acid (PGA) are derived from sodium glutamate, potassium glutamate, ammonium glutamate, calcium glutamate, magnesium glutamate or a mixture of these forms.

11. The process as claimed in claim 8, wherein in step a), the polar phase (PP) comprises, per 100% of its mass:
from 5% to 70% by mass of polyglutamic acid,
from 0.025% to 7% by mass of the crosslinking agent (XLA),
from 23% to 94.975% by mass of at least one polar solvent.

12. The process as claimed in claim 8, wherein in step a), the polar solvent (PS) is chosen from the elements of the group consisting of water, methanol, ethanol, 1-propanol, 2-propanol, isobutanol, tert-butanol, 2-methyl-2-propanol, 1-butanol, 2-butanol, acetone, dimethyl ketone, diethyl ketone, tetrahydropyran, tetrahydrofuran, 2-methyltetrahydrofuran, 1,3-dioxane and 1,4-dioxane.

13. The process as claimed in claim 8, wherein in step a), the polar phase also comprises at least one compound of formula (XIV): 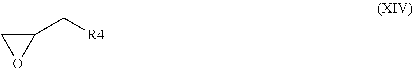
with R4 representing a linear or branched, saturated or unsaturated hydrocarbon-based radical optionally including at least one hydroxyl function and including from 6 to 22 carbon atoms.

14. The process as claimed in claim 13, wherein R4 represents a hydrocarbon-based radical chosen from the elements of the group consisting of heptyl, octyl, nonyl, decyl, undecyl, undecenyl, dodecyl, tridecyl, tetradecyl, pentadecyl, hexadecyl, heptadecyl, octadecyl, hydroxyoctadecyl, oleyl, linoleyl, linolenyl, eicosyl and dodecosyl radicals.

15. The process as claimed in claim 13, wherein the content of compound of formula (XIV) in the polar phase (PP) is, per 100% by mass of said polar phase (PP), between 0.05% and 35% by mass, it being understood that the sum of the mass proportions of the polyglutamic acid (PGA), of the crosslinking agent (XLA), of the polar solvent (PS) and of the compound of formula (XIV) is equal to 100%.

16. The process as claimed in claim 8, wherein step c) is performed with an atomizer using an air stream and a nozzle.

17. The process as claimed in claim 16, wherein the inlet temperature of the air stream is between 80 and 180° C. and preferably between 100 and 170° C.

18. A cosmetic composition for topical use (F) comprising, per 100% of its total mass, between 0.05% by mass and 10% by mass of at least one polymer (P) as defined in claim 1.
